(19) United States
(12) Patent Application Publication
(10) Pub. No.: US 2006/0241611 A1
(43) Pub. Date: Oct. 26, 2006

(54) Modular spinal implant system to assist with cervical stabilization

(76) Inventor: Frank Castro, Louisville, KY (US)

Correspondence Address: FRANK CASTRO, 834 Inspiration Way, Louisville, KY, 40245, US

(21) Appl. No.: 11/103,715

(22) Filed: Apr. 12, 2005

(51) Int. Cl. A61F 2/30

(52) U.S. Cl. 606/69

(57) ABSTRACT

A modular cervical spine implant system is described. Modularity will allow for easier insertion and greater stabilization of the cervical spine.

This nonprovisional Application claims priority from provisional patent No. 60/550,487 that was filed Mar. 8, 2004

Identification of prior art known by Applicant

U.S. PATENT DOCUMENTS

| | | | |
|---|---|---|---|
| 1 | 6,679,883 | Jan. 20, 2004 | Hawkes, et al |
| 2 | 6,652,525 | Nov. 25, 2003 | Assaker, et al |
| 3 | 6,398,783 | Jun. 4, 2002 | Michelson |
| 4 | 6,592,586 | Jul. 15, 2003 | Michelson |
| 5 | 6,454,771 | Sep. 24, 2002 | Michelson |
| 6 | 6,626,907 | Sep. 30, 2003 | Campbell et al |
| 7 | 6,306,136 | Oct. 23, 2001 | Baccelli |
| 8 | 6,231,610 | May 15, 2001 | Geisler |
| 9 | 6,224,602 | May 1, 2001 | Hayes |
| 10 | 5,904,683 | May 18, 1999 | Pohndorf et al |

### OTHER REFERENCES

1 Barrack R L. Modularity of prosthetic implants. J Am Acad Orthop Surg 1995; 3(2):79-85.
2 Castro F P Jr. Stingers, cervical cord neurapraxia, and stenosis. Clin Sports Med 2003; 22:483-492.
3 Majd M E, Vadhva M, Holt R T. Anterior cervical reconstruction using titanium cages with anterior plating. Spine 1999; 24(15): 1604-1610.
4 Park J-B, Cho Y-S, Riew K D, et al. J Bone Joint Surg 2005; 87-A:558-563.

### FIELD AND BACKGROUND OF THE INVENTION

The present invention relates to the field of cervical spine surgery. As the population ages, the prevalence of degenerative disk disease in the cervical spine has increased proportionately. A majority of spinal cord decompression surgery in the cervical spine, accomplished through an anterior surgical approach, destabilizes the spine. Thus, most surgeries that decompress the spine are coupled with some type of stabilization procedure. Cervical plate stabilization has become the standard of care in cervical arthrodesis procedures.

In 1999 Majd et al³ described a surgical technique to address the growing number of multilevel cervical reconstruction cases. Hereafter known as the cage/plate technique. A number of technical difficulties were evident with commonly used cervical plates when the cage/plate technique was used: (1) some plates were not long enough; (2) other plates were too wide; (3) most plates only allowed for screw fixation into the cephalad and caudad vertebral bodies; and, (4) plates of fixed length often necessitated sub-optimal screw trajectory.

The purposed implant system is composed of three different parts: a central stabilization bar, a fixation bar, and fixation screws. A minimum one central stabilization bar, two fixation bars, and four fixation screws are needed to stabilize one vertebral motion segment. When two or more vertebral motion segments require stabilization, additional fixation bars and screws may be used to increase the construct rigidity. This modular system will eliminate the problems commonly seen with prefabricated plates.

### SUMMARY OF THE INVENTION

The original hip replacement prosthesis, described by Sir John Charnley, was a monoblock. The stem, neck and head were created as a solid unit. This created significant difficulties at the time of surgery. Exchange of trial implants was cumbersome and often caused fractures to the host bone. An infinite combination of stem lengths, neck lengths, and head sizes required vendors to bring a large number of potential implants to surgery¹. When total hip prostheses were broken down into three component parts: stems, necks, and heads, the surgeon could determine the exact combination that would fit best for each patient.

Applying the concept of modularity to lumbar spinal surgery has also revolutionized lumbar fusion procedures. It is now time to apply the concept of modularity to cervical spine surgery. By dividing cervical plates into component parts it should allow the surgeon flexibility to accommodate the different anatomy seen at the time of surgery². Modularity will allow smaller parts to be placed through smaller skin incisions. Modular parts will also allow precise placement of fixation screws eliminating sub-optimal screw placement. The ability to increase the number of fixation points, by adding additional components, should also increase the construct rigidity and arthrodesis rate.

Spine surgeons realize that cervical stability decreases in proportion to the amount of a cervical disk removed. When more than a single disk is to be removed, a stabilization procedure is definitely indicated. Many surgeons use “static plates” such as those referenced in U.S. Pat. Nos. 6,398,783; 6,652,525; 6,592,586; 6,454,771; 6,626,907; 6,224,602; and 5,904,683 in order to stabilize the cervical spine. When part of a vertebral body is removed surgeons may use “static” or “dynamic” plates such as those described in U.S. Pat. Nos. 6,306,136 and 6,679,883. A “dynamic” plate allows for controlled shortening of the cervical plate after its implantation.

Static and dynamic cervical plates have a number of features in common. Both types of cervical plates lie anterior to the vertebral bodies they are affixed to. Both types of plates require screw fixation into the cephalad and the caudad vertebral body. This screw fixation occurs through bores or apertures in the plate. In static plates the vertical distance between the apertures, as well as the screw trajectory angle, is fixed. In dynamic plates the vertical distance between apertures may vary, both before and after implantation. As the distance between the cephalad and caudad vertebral bodies increases, the likelihood of finding a prefabricated plate of appropriate length and screw trajectory apertures decreases.

By separating the cervical plate into two components, the risks of inappropriate plate length and improper screw trajectory are minimized. The length of the central stabilization bar can be modified to accommodate various anatomic differences. Being able to place a fixation bar with apertures at any desired location should also eliminate sub-optimal screw positioning.

### BRIEF DESCRIPTION OF THE DRAWINGS

To provide a further understanding of the above objects and advantages of the present invention, a detailed description of a preferred embodiment of the present invention follows which makes reference to the appended drawings, in which:

FIG. 1 is a view of the central stabilization bar from above (anterior). The serrations (100) face anteriorly, away from the spine. The serrations run perpendicular to the long axis of the plate. The serrations are of uniform height and depth.

FIG. 2 is a side view a fixation bar. The “anterior” surface of the fixation bar is smooth and polished. The undersurface or “posterior” surface of the middle section has serrations (200) that run parallel to the long axis of the fixation bar. These “posterior” serrations will interdigitate with the “anterior” serrations of the central stabilization bar (FIG. 1 #100). The foot plate (210) on one side of the center section has an aperture that allows passage of a fixation screw (aperture not visible on side view). The foot plate (220) on the opposite side of the center section also has an aperture that allows passage of a fixation screw.

FIG. 3 is a side view another fixation bar embodiment. The “anterior” surface of the fixation bar is smooth and polished. The undersurface or “posterior” surface of the middle section has serrations (300) that run parallel to the long axis of the fixation bar. These “posterior” serrations will interdigitate with the “anterior” serrations of the central stabilization bar (FIG. 1 #100). The foot plate (310) on one side of the center section has an aperture that allows passage of a fixation screw. The foot plate (320) on the opposite side of the center section also has an aperture that allows passage of a fixation screw.

FIG. 4 is a view of the underside of a fixation bar. The serrations (400) in the middle of the fixation bar allow for interdigitation with those on the central stabilization bars (FIG. 1 #100). The foot plates (410 and 430) with apertures (420 and 440) are visible from this perspective.

FIG. 5 is a view of the anterior surface of a fixation bar. The foot plates (510 and 530) have apertures (520 and 540) that allow for passage of fixation screws.

FIG. 6 is a view of the anterior surface of another fixation bar embodiment. The foot plates (610 and 620) of this particular fixation bar has four apertures (630, 640, 650, and 660) that would allow for the passage of a maximum of four fixation screws. The undersurface or posterior surface between the two foot plates (610 and 620), identified as 600, has serrations that compliment those of the central stabilization bar (FIG. 1 #100).

### DETAILED DESCRIPTION OF A PREFERRED EMBODIMENT

Although this disclosure would enable those skilled in the art to construct and use the invention, the embodiments published herein merely exemplify the present invention.

In the most general sense, the present invention is a brace or implant that can be applied to the anterior surface of the cervical spine. It is evident that many embodiments of the current implant can be useful for accommodating the various anatomic differences seen during surgery. The implant may also assist in cervical spine surgeries by providing immediate and long-term stabilization of the spine. The reduction in component size, compared to traditional cervical plates, will also allow easier insertion of the implant through smaller surgical skin incisions.

Once assembled in situ, the implant assists in stabilizing the spinal column against numerous forces: resistance to shear stress created by lateral rotational of the cervical spine is applied; resistance to compressive loads applied to the cervical spine during cervical flexion and weight bearing in the upright position is applied; resistance to distraction (tensile) stresses are applied when the cervical spine is stretched during neck extension and/or while lying down (supine). Once assembled the implant also serves as a barrier preventing extrusion of materials, such as a bone graft or cage, from the spinal column.

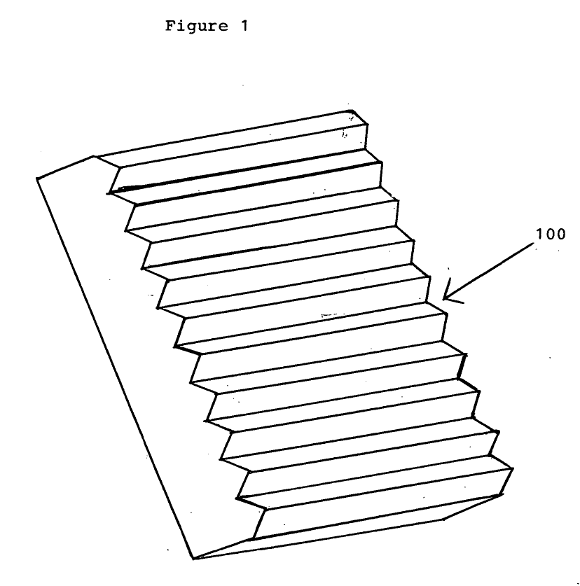

The modular feature of the current implant will accommodate to the surgical parameters, such as number of cervical bodies to be stabilized or the size of the vertebral bodies. Once assembled, the preferred embodiments of the current spinal implant are generally shaped like the capital letter “I”. All components will be manufactured with biocompatible substances, such as titanium, stainless steel, resorbable and non-resorbable polymers, or any other composition acceptable in the art. Within the scope of the present invention, it appears that the length of the central stabilization bars (FIG. 1) can be from 10 to 200 millimeters. The width of the central stabilization bars can be between 5 and 15 millimeters. The thickness of the central stabilization bars can be between 0.2 and 2 millimeters. The size of the central stabilization bar is dependent upon the anterior surface area covering the spine requiring stabilization. Prior to the surgical procedure, central stabilization bars of various lengths and widths can be available for the surgical team. The length can be varied during the surgical procedure by cutting off excess plate prior to fixation to the patient's spine. It has been shown that optimal implant positioning requires the ends of the plate to be 5 millimeter below the superior vertebral body end-plate and 5 millimeters above the interior vertebral body end-plate of the vertebral bodies being stabilized⁴.

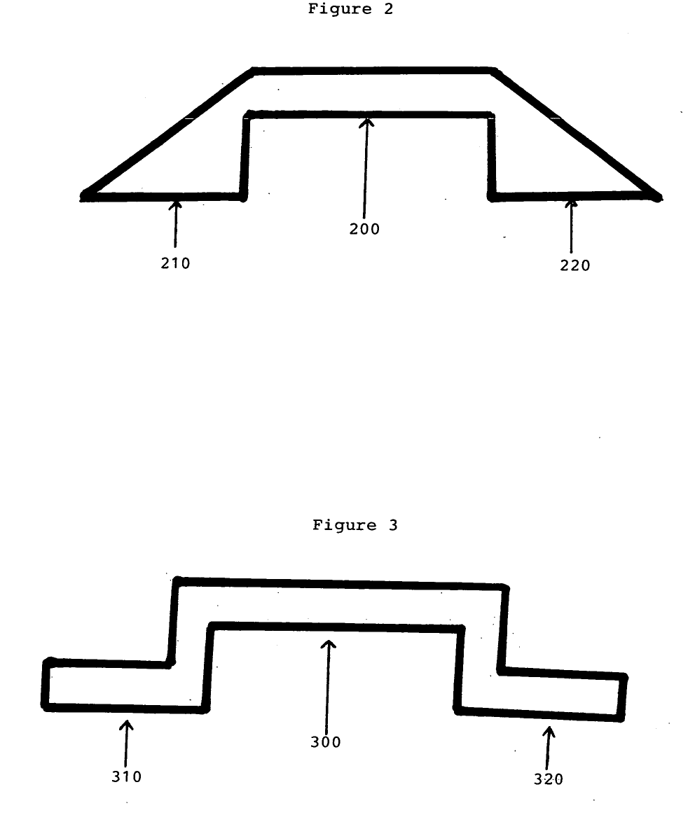

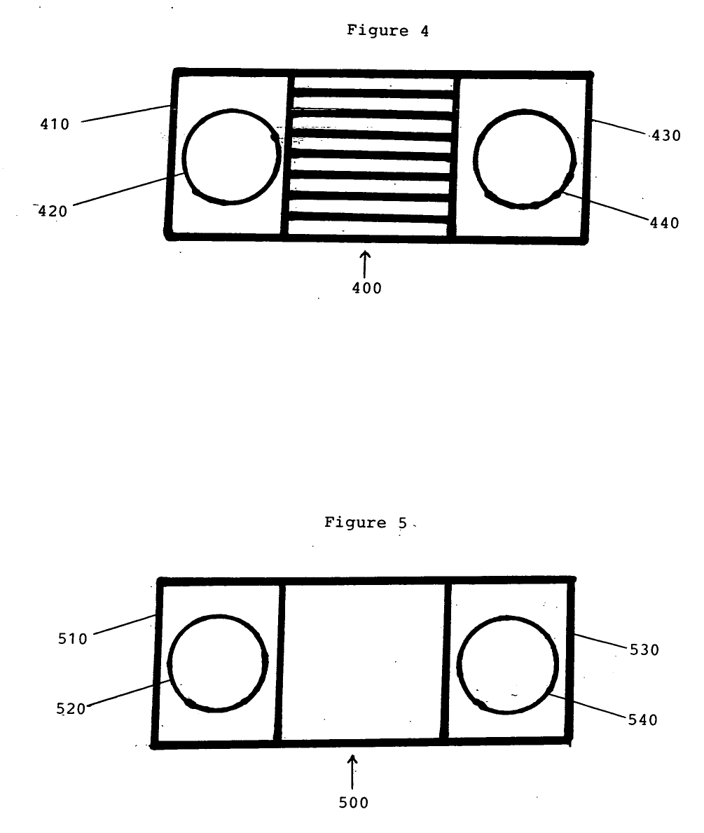

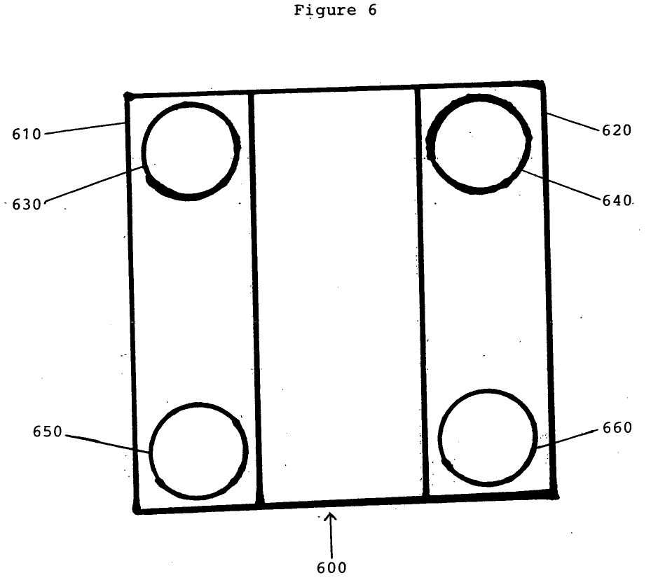

Within the scope of the present invention, it appears that the length of the fixation bars (FIGS. 2 through 6) can be from 4 to 20 millimeters. The width of the fixation bars can be between 10 and 25 millimeters. The thickness of the fixation bars can be between 0.2 and 2 millimeters. The size of the fixation bar to be used during surgery is dependent upon the anterior width of the cervical vertebral body exposed. Prior to the surgical procedure, central stabilization bars of various lengths and widths can be available for the surgical team. Since the fixation bars are placed on top of the central stabilization bar they can be positioned as far away from the ends of the construct as desired. Their positioning, independent of the central stabilization bar placement, will optimize the trajectory of the fixation screws into the vertebral bodies. Trajectory of the superior fixation screws through the superior fixation bar towards the superior end-plate and trajectory of the inferior fixation screws through the inferior fixation bar towards the inferior end-plate will optimize the construct strength. Proper cantilever biomechanics requires diverging fixation screws. Plates with apertures at fixed intervals often necessitate poor trajectory of fixation screws into the vertebral body. Convergent screw placement often result in implant failure. Additional fixation bars can also be added at the surgeon's discretion. Each fixation bar increases the strength of the stabilization construct.

Referring to FIGS. 1-6, an embodiment of the inventive cervical spine arthrodesis device is generally shown as one central stabilization bar that rests on at least two vertebral bodies. At least one fixation bar is affixed to the vertebral bodies requiring stabilization with fixation screws through the apertures. As the screws engage the vertebral bodies the serrations of the central stabilization and fixation bars interdigitate. The greater the insertion torque on the fixation screws the greater the compressive forces between the serrations of the central stabilization bar and the fixation bar.

Having disclosed the invention as required by Title 35 of the United States Code, Applicant now prays respectfully that Letters Patent be granted for his invention in accordance with the scope of the claims appended hereto.

What is claimed is:

1. A modular cervical spine implant, comprising: a stabilization bar, having a plurality of serrations thereon, wherein said stabilization bar can be positioned across one or more cervical vertebra; a fixation bar for covering said stabilization bar; said fixation bar further including:
(a) a plurality of teeth for engaging said stabilization bar's serrations;
(b) a first end having apertures for receiving a first set of fixation screws; and
(c) a second end having apertures for receiving a second set of fixation screws.

2. The device of claim 1, wherein said stabilization bar has a width of from about 5 millimeters to about 15 millimeters and a length from about 10 millimeters to about 200 millimeters.

3. The device of claim 2, wherein said stabilization bar has a thickness of from about 0.5 millimeter to about 2.0 millimeters.

4. The device of claim 3, wherein said serrations are fixed in a direction parallel said width of said stabilization bar.

5. The device of claim 4, wherein said fixation screws are from about 8 millimeters to about 20 millimeters in length and from about 1 millimeter to about 5 millimeters in diameter.

6. A method of implanting a cervical spine implant, comprising the steps of:
making a surgical opening in a patient's cervical vertebral region;
inserting a stabilization bar, wherein said stabilization bar further includes a plurality of serrations thereon;
positioning said stabilization bar over one or more cervical vertebra;
inserting a fixation bar through said surgical opening;
covering said stabilization bar with said fixation bar, wherein said fixation bar further includes a plurality of teeth for meshing with said plurality of serrations and first and second ends, each said first and second end having a plurality of apertures for receiving one or more fixation screws;
inserting one or more of said fixation screws through said surgical opening and into one or more of said plurality of apertures to anchor said cervical spine implant; and closing said surgical opening.

7. The method of claim 6 further comprising the step of shortening said stabilization bar by severing said stabilization bar along a trough between two of said plurality of said serrations.

8. A modular cervical spine implant, comprising: a rigid stabilization bar, having a plurality of serrations thereon, wherein said rigid stabilization bar can be positioned across one or more cervical vertebra; a generally C-shaped fixation bar for covering said rigid stabilization bar; said generally C-shaped fixation bar further including:
(a) a plurality of teeth positioned on an inner C side for engaging said rigid stabilization bar's serrations;
(b) a first end having a first foot plate including a plurality of apertures for receiving a first set of fixation screws; and
(c) a second end having a second foot plate including a plurality of apertures for receiving a second set of fixation screws.

9. The device of claim 8, wherein said stabilization bar has a width of from about 5 millimeters to about 15 millimeters and a length from about 10 millimeters to about 200 millimeters.

10. The device of claim 9, wherein said stabilization bar has a thickness of from about 0.5 millimeter to about 2.0 millimeters.

11. The device of claim 10, wherein said serrations are fixed in a direction parallel said width of said stabilization bar.

12. The device of claim 11, wherein said teeth are generally parallel said serrations.

13. The device of claim 12, wherein said first foot plate includes at least two apertures and wherein said second foot plate includes at least two apertures.

14. The device of claim 13, wherein said fixation screws are from about 8 millimeters to about 20 millimeters in length and from about 1 millimeter to about 5 millimeters in diameter.

15. The device of claim 13, wherein said apertures are symmetrically spaced from each other.
